(19) United States
(12) Patent Application Publication
(10) Pub. No.: US 2025/0235269 A1
(43) Pub. Date: Jul. 24, 2025

(54) Surgical Navigation Systems And Methods For Multi-Modality Tracking And Object Detection

(71) Applicant: Stryker European Operations Limited, Carrigtwohill (IE)

(72) Inventors: Emeric Umbdenstock, Freiburg (DE); Ingmar Wegner, Waldkirch (DE); Sai Manoj Prakhya, Freiburg (DE); Fabian Riegelsberger, Umkirch (DE); Philipp Schollmaier, Neuss (DE)

(73) Assignee: Stryker European Operations Limited, Carrigtwohill (IE)

(21) Appl. No.: 19/028,754

(22) Filed: Jan. 17, 2025

Related U.S. Application Data
(60) Provisional application No. 63/622,670, filed on Jan. 19, 2024.

(51) Int. Cl. A61B 34/20; G16H 40/67

(52) U.S. Cl. CPC A61B 34/20; G16H 40/67; A61B 2034/2072

(57) ABSTRACT

A surgical navigation system including a tracker, a localizer, and a controller is provided. The tracker is coupled to a surgical object and includes tracking elements arranged in a tracker geometry. The localizer is configured to track the tracker and to detect parameters of the surgical object. The controller is in communication with the localizer and is configured to detect, with the localizer, a pose of the tracker geometry. The controller is further configured to receive a first parameter of the surgical object, create a temporary descriptor describing an appearance of the surgical object, receive a second parameter of the surgical object, and update the temporary descriptor to create an updated temporary descriptor describing the appearance of the surgical object. Finally, the controller is configured to track the surgical object based on a combination of the updated temporary descriptor and the pose of the tracker geometry.

### CROSS-REFERENCE TO RELATED APPLICATIONS

The present application claims priority to and all the benefits of U.S. Provisional Patent Application No. 63/622,670, filed on Jan. 19, 2024, the entire contents of which are hereby expressly incorporated herein by reference.

### TECHNICAL FIELD

The present disclosure relates generally to systems and methods for identifying and tracking components of a surgical system.

### BACKGROUND

Surgical navigation systems assist users in tracking/locating objects in an operating room. For instance, navigation systems assist surgeons in placing surgical instruments relative to a patient's anatomy. Typically, the tool and the anatomy are tracked together with their relative movement shown on a display. Often the navigation system includes tracking devices attached to the object being tracked. A localizer cooperates with the tracking devices to determine a position of the tracking devices and, ultimately, to determine a position and/or orientation of the object. The navigation system then monitors movement of the objects via the tracking devices.

Many navigation systems rely on an unobstructed line-of-sight between the tracking device and sensors of the localizer that detect the tracking device. These navigation systems also rely on the tracking device being positioned within a field-of-view of the localizer. As a result, efforts have been undertaken to reduce the likelihood of obstructing the line-of-sight between the tracking elements and the sensors and to maintain the tracking elements within the field-of-view of the localizer. However, such navigation systems are unable to prevent obstructions to the line-of-sight that may arise during the surgical procedure as a result of the movement of objects out of the line-of-sight of the localizer, or to prevent the tracking device from moving outside of the field-of-view.

When the line-of-sight is obstructed, or when the tracking devices are outside the field-of-view, errors can occur. Typically, in this situation, navigation is discontinued and error messages are conveyed to the user until the tracking devices are detected again or the navigation system is reset. This can cause delays in surgical procedures. For instance, manipulators that rely on navigation data to autonomously position a cutting tool relative to the patient's tissue must cease operation should these errors occur. This could significantly increase the surgical procedure time, particularly if difficulty arises in restoring the line-of-sight. This is contrary to the demands of modern surgical practice that require reduced surgery times in order to reduce risks of infection and risks associated with prolonged use of anesthesia.

Many navigation systems also rely on specific geometries of the tracking devices to discern a first tracking device from a second tracking device, and thus to distinguish an object coupled to the first tracking device from another object coupled to the second tracking device. Although effective, this method relies on each tracking device have unique geometry. If two visually indistinguishable tracking devices were used with these systems, they may be unable to determine which tracking device is the first/second tracking device, or which object is coupled to the first/second tracking device. Furthermore, system complexity and cost are increased by having a unique tracker geometry for each tracker.

Thus, there is a need in the art for navigation systems and methods that overcome tracking interruptions between tracking devices and a localizer as well as issues arising from tracking two different objects using trackers with the “same” geometry.

### SUMMARY

According to a first aspect, a surgical navigation system is provided. The surgical navigation system includes a tracker coupled to a surgical object, a localizer, and a controller in communication with the localizer. The tracker includes tracking elements arranged in a tracker geometry. The localizer is configured to track the tracker and to detect parameters of the surgical object. The controller is configured to detect, with the localizer, a pose of the tracker geometry, receive a first parameter of the surgical object detected by the localizer, create, based on the first parameter, a temporary descriptor describing the appearance of the surgical object, receive a second parameter of the surgical object detected by the localizer, update, based on the second parameter, the temporary descriptor to create an updated temporary descriptor describing the appearance of the surgical object, and track the surgical object based on a combination of the updated temporary descriptor and the pose of the tracker geometry.

According to a second aspect, a surgical navigation system for tracking a surgical object during a surgical procedure is provided. The surgical navigation system includes a tracker coupled to the surgical object and including a tracker geometry; a localizer configured to detect the tracker and to detect the presence of the surgical object and a parameter of the surgical object, and a controller in communication with the localizer. The controller is configured to receive the parameter of the surgical object, create, based on the parameter of the surgical object, a temporary descriptor describing the appearance of the surgical object, detect, with the localizer, a movement of the tracker and a movement of the temporary descriptor, determine if the movement of the tracker is sufficiently related to the movement of the temporary descriptor and, if so, create a tracking entity including the tracker geometry and the temporary descriptor, and track movement of the surgical object based on movement of the tracking entity.

According to a third aspect, a surgical navigation system for tracking a surgical object during a surgical procedure is provided. The surgical navigation system includes a localizer configured to detect the presence of the surgical object and detect a parameter of the surgical object, and a controller in communication with the localizer. The controller is configured to receive, from the localizer, the parameter of the surgical object at a first time, create, based on the parameter of the surgical object received from the localizer at the first time, a temporary descriptor describing the appearance of the surgical object, receive, from the localizer, the parameter of the surgical object at a second time, determine if the parameter, as received at the first time, is different from the parameter as received at the second time, and, if so, create an updated temporary descriptor describing the appearance of the surgical object and based on the parameter as received at both the first time and the second time, and track a pose of the surgical object based on the updated temporary descriptor.

According to a fourth aspect, a surgical navigation system is provided. The surgical navigation system includes a first tracker coupled to a first surgical object and includes tracking elements arranged in a first tracker geometry, a second tracker coupled to a second surgical object and including tracking elements arranged in the first tracker geometry, a localizer configured to track the first and second trackers and to detect parameters associated with the first surgical object and second surgical object, and a controller in communication with the localizer. The controller is configured to detect, with the localizer, a pose of the first tracker and a first parameter associated with the first surgical object, create a first tracking entity based on the pose of the first tracker and the first parameter, detect, with the localizer, a pose of the second tracker and a second parameter associated with the second surgical object, create a second tracking entity based on the pose of the second tracker and the second parameter, and track poses of the first and second surgical objects based on movement of the first and second tracking entities, respectively.

According to a fifth aspect, a surgical navigation system for tracking a surgical object during a surgical procedure is provided. The surgical navigation system includes a localizer configured to detect the presence of the surgical object and detect a parameter of the surgical object, a database containing descriptors of surgical objects, and a controller in communication with the localizer. The controller is configured to receive, from the localizer, the parameter of the surgical object, compare the parameter of the surgical object to the descriptors stored in the database, determine that there are no descriptors in the database describing the appearance of the surgical object based on the comparison. In response, the controller is configured to create a temporary descriptor describing the surgical object based on the detected parameter of the surgical object and track a pose of the surgical object based on the temporary descriptor.

According to a sixth aspect, a surgical navigation system for tracking a surgical object during a surgical procedure is provided. The surgical navigation system includes a tracker coupled to the surgical object, a localizer including a first sensor and a second sensor, and a controller in communication with the localizer. The localizer is configured to detect a pose of the tracker in a first tracking modality using the first sensor and detect a parameter of the surgical object in a second tracking modality using the second sensor. The controller is configured to receive the pose of the tracker in the first imaging modality, receive the parameter of the surgical object in the second imaging modality, create, based on the detected parameter of the surgical object, a temporary descriptor describing the appearance of the surgical object, and track the surgical object based on the pose of the tracker and the temporary descriptor.

According to a seventh aspect, a surgical navigation system for tracking a surgical object during a surgical procedure is provided. The surgical navigation system includes a tracker coupled to the surgical object, a localizer including a NIR sensor and a visible light sensor, and a controller in communication with the localizer. The localizer is configured to detect a pose of the tracker in an NIR space using the NIR sensor, and detect a parameter of the surgical object in a visible light space using the visible light sensor. The controller is configured to receive the pose of the tracker in the NIR space, receive the parameter of the surgical object in the visible light space, create, based on the detected parameter of the surgical object, a temporary descriptor describing the appearance of the surgical object, determine that the pose of the tracker is no longer known in the NIR space, and, in response, track the surgical object based on the parameter of the surgical object in the visible light space.

According to an eighth aspect, a surgical navigation system for tracking a surgical object during a surgical procedure is provided. The surgical navigation system includes a tracker coupled to the surgical object, a localizer including a first sensor and a second sensor, and a controller in communication with the localizer. The localizer is configured to detect a pose of the tracker in a first tracking modality using the first sensor and detect a parameter of the surgical object in a second tracking modality using the second sensor. The controller is configured to receive the pose of the tracker detected in the first imaging modality, receive the parameter of the surgical object detected in the second imaging modality, create, based on the detected parameter of the surgical object, a temporary descriptor describing the appearance of the surgical object, create a tracking entity describing the association between the tracker and the surgical object based on the pose of the tracker and the temporary descriptor, and track the surgical object based on the pose of the tracking entity. The controller is further configured to disable the tracking entity in response to a determination that the tracker is occluded from the localizer, reenable the tracking entity in response to a determination that the tracker is no longer occluded from the localizer and that the tracker is still attached to the surgical object, and resume tracking the surgical object based on the pose of the tracking entity.

Any of the above aspects can be combined in part or in whole with any other aspect. Any of the above aspects, whether combined in part or in whole, can be further combined with any of the following implementations, in full or in part.

In some implementations, the system may be configured to learn more about the appearance of the surgical object over time. In such implementations, the first parameter may be detected by the localizer at a first time and the second parameter may be detected by the localizer at a second time. In some cases, the first time is prior to the second time. Further, the controller may be configured to detect a third parameter of the surgical object, update the updated temporary descriptor based on the third parameter, and track the pose of the surgical object based on a combination of the updated temporary descriptor and the pose of the tracker geometry. In some cases, the second parameter may be realized as a motion parameter and the controller may be configured to detect a pose change of the tracker geometry in order to create the updated temporary descriptor based on a determination that the motion parameter is sufficiently aligned with the pose change of the tracker geometry. In some cases, the controller may be configured to replace the first parameter with the second parameter in response to a determination that the second parameter is inconsistent with the first parameter.

In some implementations, at least one of the parameters may be a physical parameter of the surgical object. The physical parameter(s) may be a geometry or shape, a contour, a color, an envelope, a surface roughness, a surface marking, or a color or shading of the object. Where multiple parameters are determined, the first parameter may be a first color of the surgical object and the second parameter may be a second color of the surgical object. In some cases, the second parameter is a discoloration of the surgical object. In some implementations, at least one of the parameters may be a motion parameter of the surgical object. The motion parameter(s) may be a speed or velocity of the surgical object, an acceleration of the surgical object, a rotation of the surgical object, and/or a displacement of the surgical object.

In some implementations, the controller may be configured to create a tracking entity based on the (updated) temporary descriptor and the tracker geometry. The temporary descriptor and/or the updated temporary descriptor may include any of the physical/motion parameters. In some cases, the controller may be configured to track the surgical object based on a combination of the updated temporary descriptor and the pose of the tracker geometry by tracking the tracking entity.

In some implementations, the controller may be configured to detect an environmental condition and to normalize the detected parameters according to the environmental condition. The environmental condition may be an illuminance of the surgical object, an illuminance of the tracking geometry, and/or a general illuminance of a space within a line of sight of the localizer.

In some implementations, the controller may be configured to operate in multiple states. In such implementations, the controller may be configured to operate in a first state in which the controller associates the tracker geometry with the surgical object upon a triggering event, and a second state in which the controller automatically associates the tracker geometry with the surgical object. The triggering event may be an input from a user, or the surgical object being in a predefined pose.

In some implementations, the localizer may be capable of detecting multiple modes of light. In such implementations, the localizer may include a first sensor configured to detect visible light, and second sensor configured to detect infrared or near-infrared light. The pose of the tracker geometry may be detected by the second sensor and/or the first parameter may be detected by the first sensor. In these implementations, the controller may be configured to detect the pose of the tracker geometry in a first coordinate system and the first parameter in a second coordinate system. The controller may be configured to register at least one of the first and second coordinate systems to a third coordinate system, and/or the controller may be configured to register one of the first and second coordinate systems to the other of the first and second coordinate systems.

In some implementations, the controller may be configured to determine that the pose of the tracker geometry is not detectable by the localizer. In such implementations, the controller may be configured to track the pose of the surgical object based solely on the updated temporary descriptor in response to determining that the pose of the tracker geometry is not detectable by the localizer. In some cases, the controller may be configured to determine that the pose of the tracker geometry is detectable by the localizer after determining that the pose of the tracker geometry is not detectable by the localizer. The controller may be configured to track the pose of the surgical object based on at least one of the updated temporary descriptor and the pose of the tracker geometry in response to determining that the pose of the tracker geometry is detectable by the localizer. Further, the controller may be configured to register or re-register the tracker to the surgical object by comparing a new parameter of the surgical object to the updated temporary descriptor.

In some implementations, the controller may utilize a machine learning module. For example, the controller may provide the first and second parameters to a machine learning module, and the machine learning module may output a calculated parameter of the surgical object based on the first and second parameters. In some cases, the controller may be configured to update the updated temporary descriptor based on the calculated parameter to create a learned temporary descriptor describing the association between the surgical object and the detected tracker geometry. The system may track the pose of the surgical object based on a combination of the learned temporary descriptor and the pose of the tracker geometry. The controller may be configured to receive a third parameter of the surgical object, compare the calculated parameter to the third parameter, update the learned temporary descriptor based on the comparison to create an updated learned temporary descriptor describing the association between the surgical object and the detected tracker geometry. The system may track the pose of the surgical object based on a combination of the updated learned temporary descriptor and the pose of the tracker geometry.

In some implementations, the system may be configured to track multiple surgical objects by tracking multiple trackers. In such implementations, the tracker may be realized as a first tracker and the surgical object may be realized as a first surgical object. In some cases, a second tracker may be coupled to a second surgical object and including tracking elements arranged in a tracker geometry that is identical to the tracker geometry of the first tracker. In order to distinguish the first tracker from the second tracker and vice versa, the localizer may be configured to track the first and second trackers and to detect parameters associated with the first surgical object and second surgical object. Further, the controller may be configured to detect, with the localizer, a pose of the first tracker and a first parameter associated with the first surgical object, create a first tracking entity based on the pose of the first tracker and the first parameter, detect, with the localizer, a pose of the second tracker and a second parameter associated with the second surgical object, create a second tracking entity based on the pose of the second tracker and the second parameter, and track poses of the first and second surgical objects based on movement of the first and second tracking entities, respectively.

In some implementations, the system may include a database containing descriptors of surgical objects and confirm that the surgical object detected by the localizer does not match previously detected surgical objects. In such implementations, the controller may be configured to compare the first parameter of the surgical object to the descriptors stored in the database, determine that there are no descriptors in the database describing the appearance of the surgical object based on the comparison, and, in response, create the temporary descriptor describing the surgical object based on the detected parameter of the surgical object. In some cases, the updated temporary descriptor may be realized as a database entry containing at least the first and second parameters.

In some implementations, the system may include a display in communication with the controller which may be configured to depict the surgical object relative to a surgical target. In some cases, the surgical object may be represented by a computer graphic and the computer graphic may be depicted relative to the surgical target. The computer graphic may be based on one of the temporary descriptor(s) and the parameter(s). The computer graphic may include a unique identifier which identifies the surgical object

In some implementations, the system may be configured to make determinations regarding surgical objects and/or trackers which have become occluded from the localizer. For example, the controller may be configured to determine that the tracker is still associated with the surgical object by comparing a new parameter of the surgical object to the temporary descriptor. In such an example, the parameter of the surgical object may be realized as an initial parameter and the controller may be configured to compare the new parameter to the temporary descriptor by comparing the new parameter to the initial parameter.

The operations described in reference to the controller, such as according to any combination of the aspects and implementations described herein, may be implemented as a computer program product or instructions stored on a non-transitory computer readable medium. In the case of the computer program product, the computer program product may be configured to be executed by the controller so as to cause the controller to carry out the operations. In the case of the non-transitory computer readable medium, the non-transitory computer readable medium may be connected to a controller, and the instructions stored on the non-transitory computer readable medium may be configured to cause the controller to carry out the operations.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a perspective view of one implementation of a surgical system comprising a robotic manipulator and a navigation system including a localizer.

FIG. 2A is a perspective views of the surgical system of FIG. 1 along with tracking entities created by the navigation system, according to one implementation.

FIG. 2B is a perspective view of the surgical system of FIG. 1 as represented by the tracking entities of FIG. 2A, according to one implementation.

FIG. 3 is a block diagram of one implementation of a software suite that can be utilized by the navigation system of FIG. 1.

FIG. 4 is a flow diagram of a method of tracking objects in an operating room, according to one implementation.

FIG. 5 is a flow diagram of a tracking manager process for managing tracking entities, according to one implementation.

FIG. 6 is a schematic representation of a tracking entity as created by the navigation system, according to one implementation.

FIG. 7 is a schematic representation of various tracking entities as created by the navigation system, according to one implementation.

FIG. 8 is a flow diagram of a navigation manager process for tracking surgical objects, according to one implementation.

FIG. 9 is a flow diagram of a method of tracking objects in an operating room, according to one implementation.

### DETAILED DESCRIPTION

### I. Example System Overview

Referring to the Figures, wherein like numerals indicate like or corresponding parts throughout the several views, a surgical navigation system (hereinafter “system”) and method for operating the same are shown throughout.

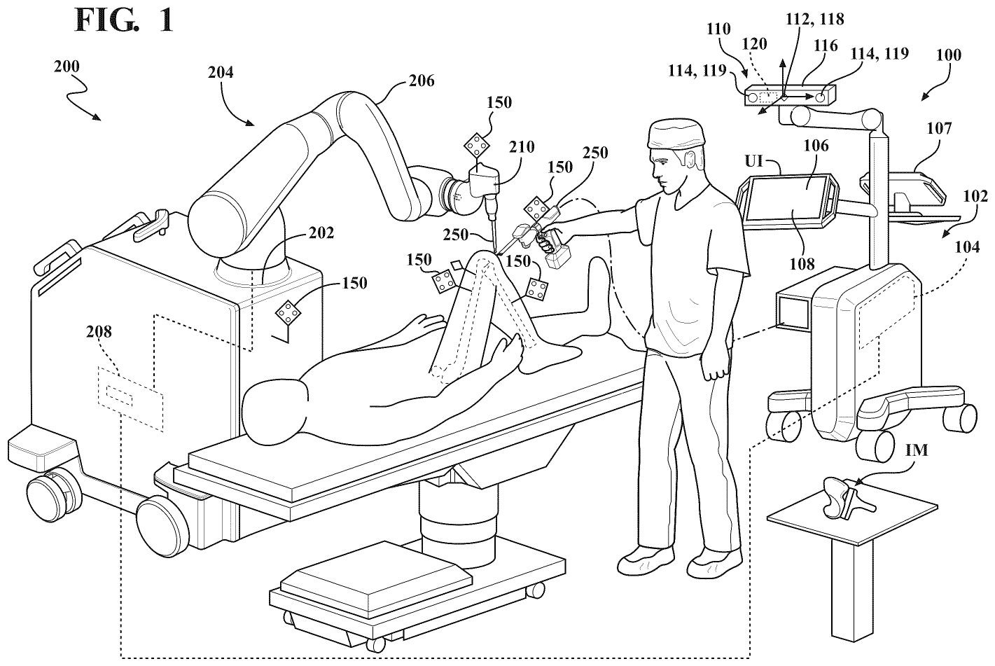

Referring to FIG. 1, an example configuration of an operation room or surgical suite for performing a medical procedure on a patient is shown. The illustrated configuration includes a surgical navigation system 100, a surgical robot 200, a surgical instrument 250, and an implant IM to be implanted into the patient. The surgical navigation system 100 is set up to track movement of various objects in the operating room. Such objects include, for example, the patient, the surgical robot 200, and/or the surgical instrument 250, among other objects. The surgical navigation system 100 tracks these objects for purposes of displaying their relative positions and orientations to the surgeon and, in some cases, for purposes of controlling or constraining movement of the surgical instrument 250 relative to virtual cutting boundaries associated with the patient.

The surgical navigation system 100 may include a computer cart assembly 102 that houses a navigation controller 104. A navigation interface is in operative communication with the navigation controller 104. The navigation interface includes a first display 106 adapted to be situated outside of the sterile field and a second display 107 adapted to be situated inside the sterile field. The displays 106, 107 are adjustably mounted to the computer cart assembly 102. First and second input devices (not shown) such as a keyboard and mouse can be used to input information into the navigation controller 104 or otherwise select/control certain aspects of the navigation controller 104. Other input devices are contemplated including a touch screen 108, gesture control, or voice-activation. The displays can be implemented as head-mounted displays configured for extended reality or augmented reality and adapted to display any of the graphics or imagery described herein in manner that is overlaid or superimposed over real-world views or video.

Further, the navigation system 100 includes a localizer 110 in communication with the navigation controller 104. In the illustrated implementation, the localizer 110 is a multi-modality localizer and includes an optical (video) camera unit 112 and an infrared and/or near-infrared (NIR) sensor unit 114. The optical camera unit 112 includes one or more sensors 118 that are adapted to sense light in the visible spectrum and may be configured as a video camera or machine vision or computer vision system. The visible light sensors 118 are configured to detect color and or produce data that can be used to create depth maps. The infrared sensor unit 114 may include one or more sensors 119 that are adapted to sense light in the infrared or near-infrared spectrum. The localizer 110 may include illuminators that are configured to radiate infrared light into the surgical field to enable the infrared sensors 119 to detect reflected or backscatter radiation. An outer casing 116 that houses the optical camera unit 112 and the infrared sensor unit 114.

The localizer 110 is in communication with the navigation controller 104. In some implementations, a camera controller 120 facilitates communication between the sensors 118, 119 and the navigation controller 104 through either a wired or a wireless connection (not shown). In other implementations, the sensors 118, 119 may communicate directly with the navigation controller 104. Processing of the signals from the visible light sensor(s) 118 and the IR sensor(s) 119 may occur at the navigation controller 104 for processing both navigation and machine vision information. One example of the navigation system 100 is described in U.S. Pat. No. 9,008,757, entitled, “Navigation System Including Optical and Non-Optical Sensors,” hereby incorporated by reference.

The navigation controller 104 can be a personal computer or laptop computer. The navigation controller 104 may have the display 106, central processing unit (CPU) and/or other processors, memory (not shown), and storage (not shown). The navigation controller 104 may be loaded with software which converts the signals received from the camera unit 112 and the infrared sensor unit 114 into data representative of the position and orientation of the objects being tracked. Additionally, the software converts the signals received from the camera unit 112 into data that can identify the objects, such as through object recognition from the optical camera unit 112. Position and orientation signals and/or data are transmitted to the navigation controller 104 for purposes of tracking objects. In an alternative, all of the computer processing components and functionality may be integrated into a single processing units or may be distributed between or among multiple processing units. Moreover, although described as taking place at a particular computer or controller in the present disclosure, it will be appreciated by one of skill in the art that any processing tasks may take place or be performed by other computers or controllers. The computer cart assembly 102, display 106, and camera unit 112 may be like those described in U.S. Pat. No. 7,725,162 to Malackowski, et al. issued on May 25, 2010, entitled “Surgery System,” hereby incorporated by reference.

The surgical navigation system 100 may be used to track the poses of a plurality of tracking devices 150, herein referred to as trackers 150. In the illustrated implementation, one tracker 150 is coupled to a first anatomical position of the patient, another tracker 150 is coupled to a second anatomical position of the patient, another tracker 150 is coupled to the surgical instrument 250, another tracker 150 is coupled to the surgical robot 200 (or the instrument 250 coupled thereto), and other trackers 150 are contemplated.

The trackers 150 may be active trackers or passive trackers. Active trackers require a power source and have an array of fiducials (also referred to as tracking elements or markers) that actively generate and emit radiation in a wavelength detectable by the visible light sensor 118. The fiducials of an active tracker may be a light emitting diode (LED), including, for example, an infrared LED. The array of active fiducials may be “always on” or may be operative to selectively fire, that is emit radiation, according to and in response to commands from the surgical navigation system 100. In such selective-fire active trackers, the tracker may communicate by way of a wired or a wireless connection with the navigation controller 104 of surgical navigation system 100. In other examples, the active trackers can include active electromagnetic elements, active radio-frequency elements, and the like.

In alternative implementations, the trackers 150 may include passive trackers. The active tracker may be battery powered with an internal battery or may have leads to receive power through the navigation controller 104, which may receive external power. The passive tracker array typically does not require a power source. Passive trackers can include barcodes, QR codes, or any computer-detectable pattern. Passive trackers can include passive reflective markers, radio-opaque markers, passive electromagnetic elements, passive radio-frequency elements, and the like.

Further, the trackers 150 may each include a tracker geometry. For example, where the tracker(s) 150 includes an array of optical elements, these optical elements may be arranged relative to one another to form the tracker geometry. In some implementations, each tracker 150 has a common tracker geometry. In other implementations, each tracker 150 has a unique tracker geometry. In further implementations, multiple trackers 150 may each have a common tracker geometry, while other trackers 150 may have unique tracker geometries which are different from the common tracker geometry.

In some examples, the navigation system 100 and/or the localizer 110 are radio frequency (RF) based. For example, the navigation system 100 may comprise an RF transceiver coupled to the navigation controller 104. Here, the trackers 150 may comprise RF emitters or transponders, which may be passive or may be actively energized. The RF transceiver transmits an RF tracking signal, and the RF emitters respond with RF signals such that tracked states are communicated to (or interpreted by) the navigation controller 104. The RF signals may be of any suitable frequency. The RF transceiver may be positioned at any suitable location to track the objects using RF signals effectively. Furthermore, examples of RF-based navigation systems may have structural configurations that are different than the navigation system 100 illustrated throughout the drawings.

In some examples, the navigation system 100 and/or localizer 110 are electromagnetically (EM) based. For example, the navigation system 100 may comprise an EM transceiver coupled to the navigation controller 104. Here, the trackers 150 may comprise EM components attached thereto (e.g., various types of magnetic trackers, electromagnetic trackers, inductive trackers, and the like), which may be passive or may be actively energized. The EM transceiver generates an EM field, and the EM components respond with EM signals such that tracked states are communicated to (or interpreted by) the navigation controller 104. The navigation controller 104 may analyze the received EM signals to associate relative states thereto. Here too, examples of EM-based navigation systems may have structural configurations that are different than the navigation system 100 illustrated throughout the drawings.

In some examples, the navigation system 100 and/or the localizer 110 could be based on one or more other types of tracking systems. For example, an ultrasound-based tracking system coupled to the navigation controller 104 could be provided to facilitate acquisition of ultrasound images of markers that define trackable features 150 such that tracked states are communicated to (or interpreted by) the navigation controller 104 based on the ultrasound images. By way of further example, a fluoroscopy-based imaging system (e.g., a C-arm) coupled to the navigation controller 104 could be provided to facilitate acquisition of X-ray images of radio-opaque markers that define trackable features such that tracked states are communicated to (or interpreted by) the navigation controller 104 based on the X-ray images.

Furthermore, in some examples, a machine-vision tracking system (e.g., one or more charge-coupled devices) coupled to the navigation controller 104 could be provided to facilitate acquiring 2D and/or 3D machine-vision images of structural features that define trackable features 150 such that tracked states are communicated to (or interpreted by) the navigation controller 104 based on the machine-vision images. The ultrasound, X-ray, and/or machine-vision images may be 2D, 3D, or a combination thereof, and may be processed by the navigation controller 104 in near real-time to determine the tracked states of the trackers 150.

Various types of tracking and/or imaging systems could define the localizer 110 and/or form a part of the navigation system 100 without departing from the scope of the present disclosure. Furthermore, the navigation system 100 and/or localizer 110 may have other suitable components or structure not specifically recited herein, and the various techniques, methods, and/or components described herein with respect to the optically based navigation system 100 shown throughout the drawings may be implemented or provided for any of the other examples of the navigation system 100 described herein. For example, the navigation system 100 may utilize solely inertial tracking and/or combinations of different tracking techniques, sensors, and the like. Other configurations are contemplated.

The navigation system 100 may be used to track the surgical robot 200 as noted above. In some implementations, the surgical robot 200 includes a base 202 and a manipulator 204 including a plurality of links and joints. The base 202 may be fixed to a point in the operating room, such as the operating table. Alternatively, the base 202 may be readily movable such that the surgical robot can be repositioned in the operating room. In one example, the surgical robot 200 can have a configuration such as the robotic manipulator described in U.S. Pat. No. 10,327,849, entitled “Robotic System and Method for Backdriving the Same”, the contents of which are hereby incorporated by reference in its entirety.

The surgical robot 200 may house a manipulator controller 208, or other type of control unit. The manipulator controller 208 may comprise one or more computers, or any other suitable form of controller that directs the motion of the manipulator 204 and/or the base 202. The manipulator controller 208 may have a central processing unit (CPU) and/or other processors, memory, and storage. The processors could include one or more processors to control operation of the manipulator 204. The processors can be any type of microprocessor, multi-processor, and/or multi-core processing system. The manipulator controller 208 may additionally, or alternatively, comprise one or more microcontrollers, field programmable gate arrays, systems on a chip, discrete circuitry, and/or other suitable hardware, software, or firmware that is capable of carrying out the functions described herein. The term processor is not intended to limit any implementation to a single processor. The surgical robot 200 may also comprise a user interface UI with one or more displays and/or input devices (e.g., push buttons, keyboard, mouse, microphone (voice-activation), gesture control devices, touchscreens, etc.).

The surgical robot 200 may be used to control the surgical instrument 250. To that end, the surgical robot 200 may include an end effector 210 configured to couple the surgical instrument 250 to the manipulator 204. In some implementations, the manipulator 204 and the instrument 250 may be arranged like that shown in U.S. Pat. No. 9,566,121, filed on Mar. 15, 2014, entitled, “End Effector of a Surgical Robotic Manipulator,” hereby incorporated by reference. In other implementations, the surgical instrument 250 is attached to the manipulator 204 as shown in U.S. Pat. No. 9,119,655, issued Sep. 1, 2015, entitled, “Surgical Manipulator Capable of Controlling a Surgical Instrument in Multiple Modes”, the disclosure of which is hereby incorporated by reference.

### II. Example Tracking Methods

As described above, each of the respective trackers 150 may be active and/or passive. Ordinarily, the localizer 110 would distinguish the trackers 150 (and thus the object to which the tracker is attached) from one another based on the specifics of each tracker 150. For example, the localizer 110 may distinguish one tracker from another based on the shape of the trackers 150 and/or the arrangement of tracking elements (i.e., the unique tracker geometry). In the present system 100, however, each tracker 150 may have the same shape and/or the same arrangement of tracking elements. As such, the localizer 110 may not be able to rely solely on the details of the trackers 150 to identify/track the object to which the trackers 150 are attached.

Instead of relying on the details of the trackers to identify/track the objects in the operating room, the navigation system 100 may utilize the localizer 110 to determine parameters of the objects coupled to the trackers 150 to distinguish the objects from one another such that each object can be independently identified and tracked by the system 100. The parameters of the objects may include physical parameters, such as a geometry or shape, a contour, an envelope, a surface roughness, a surface marking, or a color or shading of the object. The parameters may also include a motion parameter of the object, such as a speed or velocity, acceleration, a rotation, and/or a displacement of the surgical object.

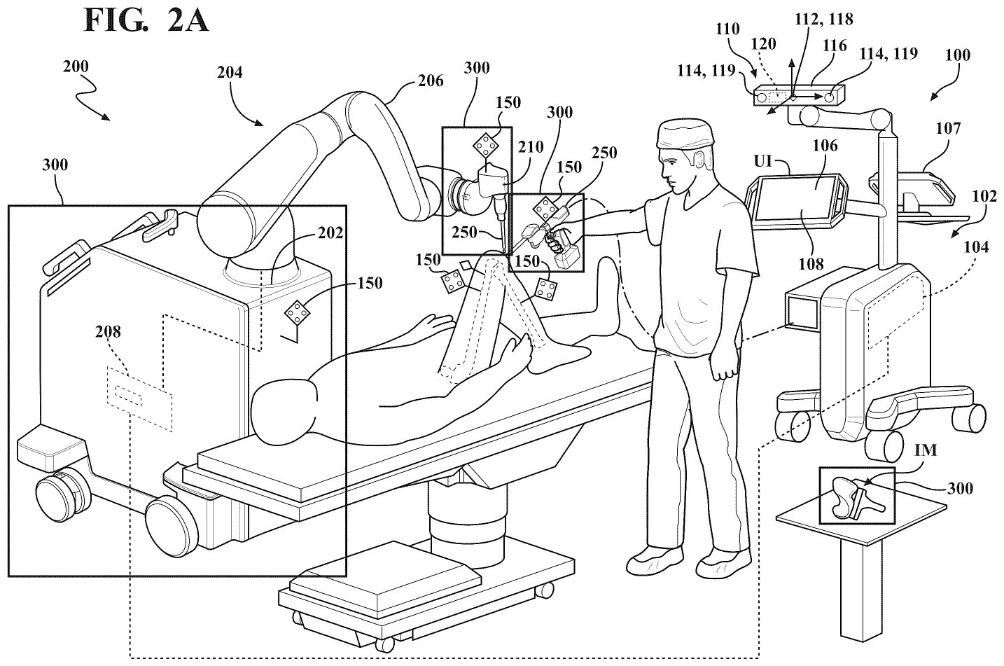

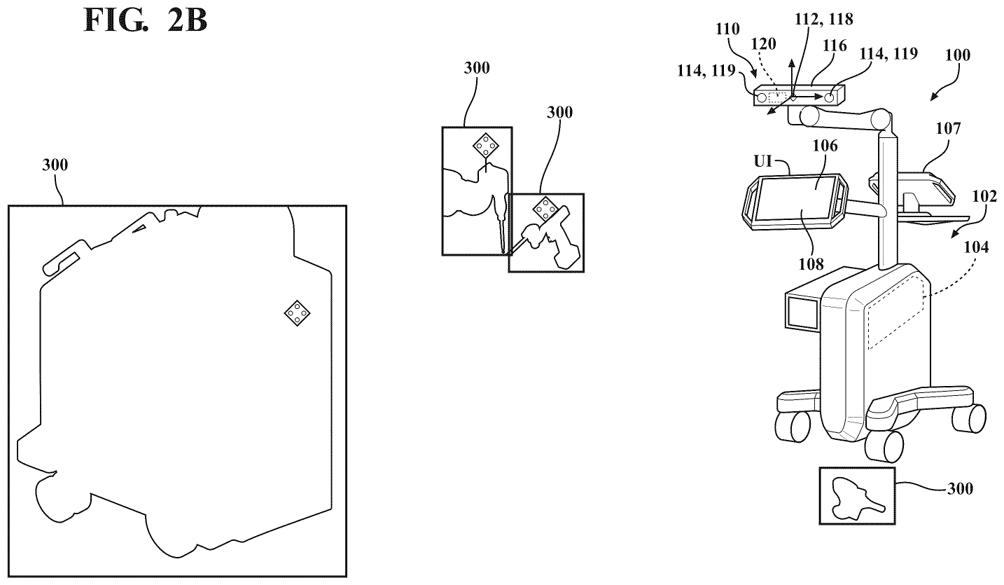

Referring now to FIGS. 2A and 2B, the operating room of FIG. 1 is shown along with each of the systems 100, 200, the instrument 250, and the implant IM. Additionally, FIGS. 2A and 2B include various bounding boxes surrounding each of the elements 200, 250, IM (aside from the navigation system 100). These bounding boxes are abstract visualizations meant to depict how the navigation system 100 sees each element 200, 250, IM. More specifically, each bounding box represents a tracking entity 300 based on the parameters of the object and the tracker 150 attached thereto. The tracking entity 300 describes the association between the object and the tracker 150. More specifically, the tracking entity 300 includes contextual information associated with the tracker 150 such that the tracker 150 can be distinguished from other trackers 150. For example, where the tracker 150 is coupled to the object, the contextual information includes a description of the object as detected by the visible light sensors 118. For the objects that do not include a tracker 150, such as the implant IM, the tracking entity 300 is based solely on the parameters of the object.

The tracking entities 300 are created by the navigation system 100 and allow the system 100 to track objects visible to the localizer 110. Unlike some other navigation systems and methods, the present system 100 does not rely solely on prestored data to identify and track the objects in the operating room. Instead, the system 100 relies on the tracking entity 300 to track the object associated with that tracking entity 300. The tracking entity 300 may be created when the object is moved into view of the localizer 110. For example, if the surgical instrument 250 is moved into view of the localizer 110 during a procedure, the navigation system 100 creates the tracking entity 300 for the instrument 250 intraoperatively. This allows the navigation system 100 to identify and track any object that may be discerned by the localizer 110, rather than only the objects known to the system 100 based on prestored data.

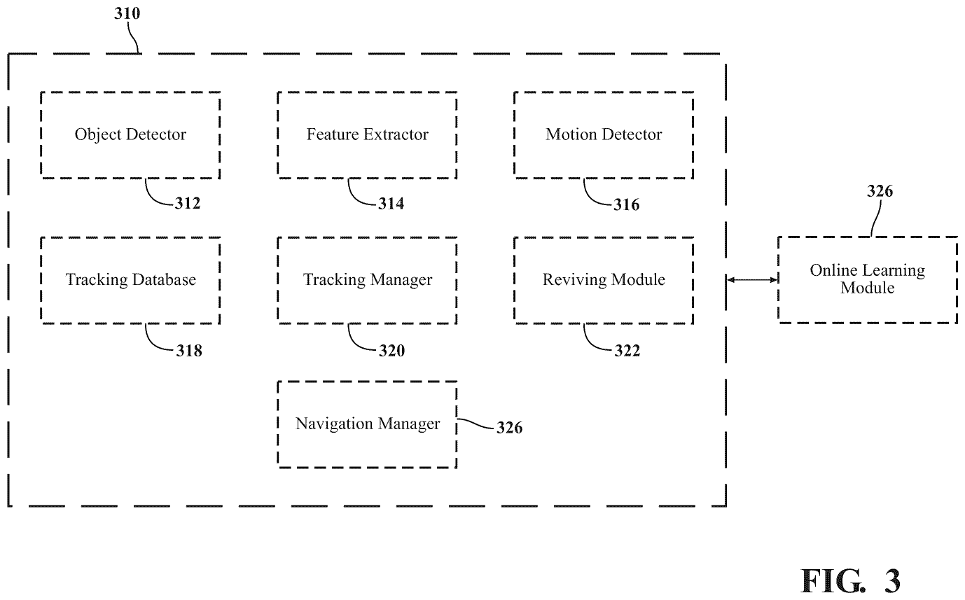

Referring to FIG. 3, an example of a software suite 310 operable by the navigation controller 104 is shown. The illustrated software suite 310 is employed by the navigation controller 104 to create the tracking entities 300 and track the objects in the operating room using said entities 300. The software suite 310 may include an object detector 312, a motion detector 316, a feature extractor 314, a tracking database 318, a tracking manager 320, a reviving module 322, and a navigation manager 324. Although the software suite 310 is illustrated and described as part of the navigation controller 104, it is further contemplated that any element of the software suite 310 may be incorporated into other computing components. The software suite 310, or a part thereof, may also be present in a remote computing component. For example, part of the software suite 310 may be present on the navigation controller 104, while the rest of the software suite 310 may reside in a cloud computing environment. The software suite 310 may also include and/or communicate with an online learning module 326. Similar to the software suite 310, the online learning module 326 may be present on the navigation controller 104 (or other element of the system 100). Alternatively, the module 326 may be present in a remote computing component.

Each element of the software suite 310 may be used to create and track the tracking entity/entities 300. The object detector 312 applies an object detection algorithm to the images captured by the localizer 110 in order to detect objects present in the operating room (i.e., present in the images). In one implementation, the object detector 312 determines an intersection over union metric and compares the metric to a confidence threshold to decide whether a feature present in the image captured by the localizer 110 is an object for the purpose of creating a tracking entity 300. In such an implementation, any feature in the image with a confidence score higher than the confidence threshold is considered an object by the object detector 312.

The motion detector 316 and the feature extractor 314 are both used to determine the parameters of the detected objects from the object detector 312. The motion detector 316 may use any suitable method to track movement of the objects in the operating room. For example, the motion detector 316 may utilize Kalman filtering to estimate and/or predict motion associated with the objects. The feature extractor 314, on the other hand, may use any suitable method to extract features of the objects in the operating room from the images captured by the localizer 110.

The navigation controller 104 writes entries to the tracking database 318 to create tracking entities 300 based on the objects detected by the object detector 312. Alternatively, the controller 104 may determine that a tracking entity already exists for the object and recall and/or modify entry in the database 318 which is associated with the existing tracking entity 300. Further, the navigation controller 104 may call the tracking manager 320, the reviving module 322 to control the tracking entities 300. The tracking manager 320 may output, modify, deactivate, and/or delete the tracking entities 300 based on the objects and associated parameter(s) detected in the images captured by the localizer 110. The reviving module 322 may reactivate previously created tracking entities 300 based on the same.

The navigation manager 324 may be called by the navigation controller 104 during navigation of surgical objects such as the surgical robot 200, the surgical instrument 250, and/or other objects present in the operating room. As described in more detail below, the navigation manager 324 is generally configured to control how the system 100 tracks the surgical objects using the visible light and IR sensors 118, 119 of the localizer 110.

The tracking database 318 has been described as having tracking entities 300 stored therein. In some implementations, the tracking entities 300 include a temporary descriptor which describes the appearance of the object according to the localizer 110. In such an implementation, the tracking entities 300 each contain the respective temporary descriptor and the respective tracker 150. If the object does not have the tracker 150 coupled thereto, the tracking entity 300 associated with that object does not include the tracker 150. The temporary descriptor generally includes all of the parameters of the object as detected by the localizer 110. In one example, the temporary descriptor includes a two/three-dimensional model of the object created from the shape of the object as captured by the localizer 110. In such an example, the system 100 knows that the object is present in the image data if the tracker 150 coupled to the object and the parameters of the object match that of the temporary descriptor. Further, the temporary descriptor may be updated as the system 100 determines other parameters of said object.

In some implementations, the tracking database 318 is structured to store each tracking entity 300 as an entry to the database 318. Here, the temporary descriptor and the tracker are each stored as sub-entries of the entry embodying the tracking entity 300, and the parameters are stored as sub-entries of the temporary descriptor sub-entry. For example, the tracking database 318 may contain a series of rows and columns. In this example, a row may represent the tracking entity 300. Included in that row, a first column may contain a unique ID, a second column may contain a representation of the tracker 150 (e.g., the tracker geometry), and a third column may contain the temporary descriptor (and thus the parameters) of the object.

As described above, each temporary descriptor includes at least one parameter of the object associated therewith and represents the description of each object as understood by the navigation system 100. The navigation system 100 uses the temporary descriptors to track the objects and to determine which object each of the trackers 150 are coupled to. Although the tracking entities 300 are shown in the figures as including parameters which only describe the shape of the object, this is merely for ease and clarity of illustration. The tracking entities/temporary descriptors can instead include other parameters of the object which are detectable by the localizer 110.

For example, where the object is the surgical instrument 250, the temporary descriptor may include parameters such as the outer contour/shape of the instrument 250, the color(s) of the instrument 250, a surface marking on a housing of the instrument 250, and a location of the instrument 250. These parameters may be continuously updated as the localizer 110 detects new and/or changed parameters. In the present example, the navigation system 100 may have initially creating the temporary descriptor with only one of the above parameters, such as the outer contour/shape of the instrument 250. Subsequently, the navigation system 100 may have detected the color and surface marking on the housing of the instrument 250 and added these parameters to the temporary descriptor to create an updated temporary descriptor. Following that, the system 100 may detect the location of the instrument 250 as, for example, in the middle of the operating room as shown in FIGS. 1-2B and add the location as another parameter to the (updated) temporary descriptor. If a surgeon were to walk between the localizer 110 and the instrument 250 such that the instrument 250 became momentarily occluded from view of the localizer 110, the system 100 could reidentify the instrument 250 by comparing the parameters of the instrument 250 to the parameters of the (updated) temporary descriptor. If the instrument 250 did not change and/or move while it was momentarily occluded from view of the localizer 110, the system 100 may determine that all/enough of the parameters of the instrument 250 match the temporary descriptor previously associated with the instrument 250 and reassociate the (updated) temporary descriptor with the instrument 250.

The temporary descriptor of the surgical instrument 250, or any other object detectable by the localizer 110, may also include motion parameters of the instrument 250. Continuing with the example above, and referring to FIGS. 1-2B, the surgeon may move the instrument 250 behind the patient such that the localizer 110 loses sight of the instrument 250 as it is moved downwards. At this point, the temporary descriptor may include a last known location of the instrument 250 and a downward velocity of the instrument 250. If the surgeon moves the instrument 250 back upward and within view of the localizer 110, the localizer 110 may detect the new location and/or velocity of the instrument 250 and the system may determine that the instrument 250 is the same one that the localizer 110 previously lost sight of and reassociate the temporary descriptor with the instrument 250. This may be done by comparing the new location/velocity detected when the instrument 250 moved back into view of the localizer 110 to the most recent location parameter of the temporary descriptor.

The temporary descriptor of the object may include parameters detected by the localizer 110 at different times, such as like the different motion parameters described in the above example. In the above example, the shape of the instrument 250 may have been detected at a first time, and the surface markings may have been detected at a second time which was after the first time. The system 100 may even replace some of the parameters included in the temporary descriptor of the object if the system 100 determines that the object has changed over time. For example, the localizer 110 may detect that the instrument 250 is mostly white at the first time. At the second time, however, the localizer 110 may detect that the instrument is now half red and half white (e.g., due to blood covering part of the instrument 250). The system 100 may determine that these two parameters are inconsistent with one another and replace the mostly white parameter of the associated temporary descriptor with the half red half white parameter detected at the second time.

The online learning module 326 may contain a machine learning algorithm configured to be trained on image data collected by the navigation system 100. As a result of this training, the online learning module 326 may be able to calculate additional parameters of the object according to previously detected parameters of the object. These additional parameters generated by the online learning module 326 are herein referred to as calculated parameters. In one example, the localizer 110 may detect a first parameter which includes the shape of the instrument 250 from a first perspective. The first parameter may be input to the online learning module 326 and the machine learning algorithm may generate a calculated parameter corresponding to the shape of the instrument 250 from a second perspective. In another example, the localizer 110 may detect the first and second parameters as the shape of the instrument 250 from the first perspective and the color of a side of the instrument 250 facing the localizer 110. These parameters may be input to the machine learning algorithm, and the algorithm may generate a first calculated parameter as the shape of the instrument 250 from the second perspective and a second calculated parameter as a color of an opposite side of the instrument 250 (which is not facing the localizer 110). These calculated parameter(s) may be added to the (potentially previously updated) temporary descriptor of the instrument 250 to create a learned temporary descriptor. Just like other parameters, the calculated parameters may be updated/changed by the system 100 if new parameters are detected which are inconsistent with the calculated parameters.

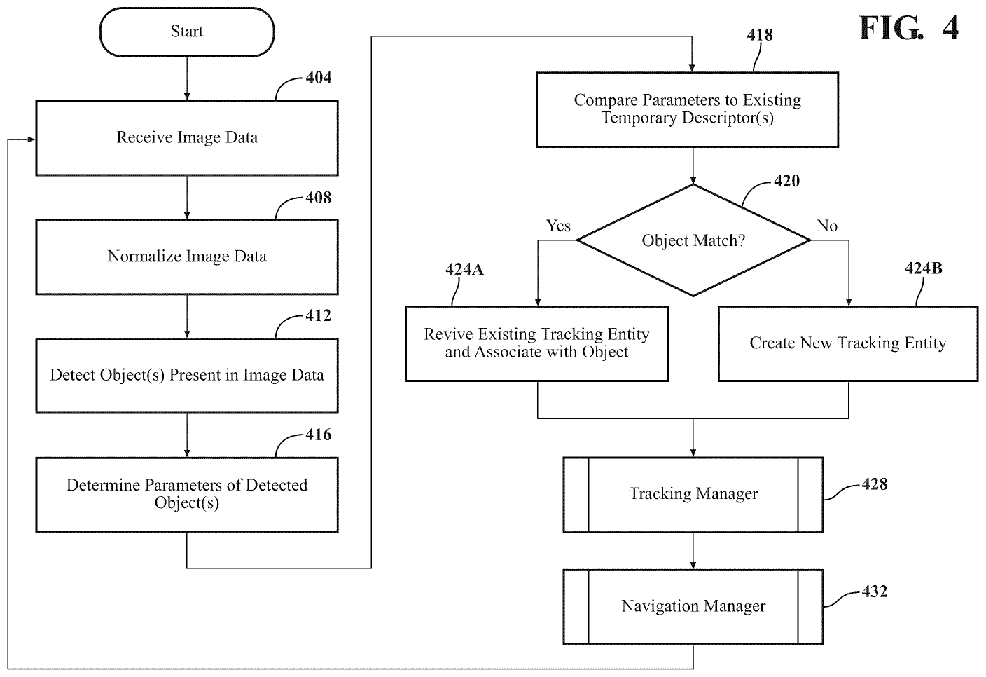

Referring to FIG. 4, an example method 400 of tracking objects in the operating room is depicted. The method 400 may be carried out entirely by the navigation controller 104, partially by the navigation controller 104 and partially by other elements of the system 100, entirely by elements of the system 100 other than the navigation controller 104, partially by the navigation controller 104 and partially by a remote computing component, or entirely by a remote computing component. For clarity and ease of description, the method 400 is described as being carried out by the system 100 generally.

The method 400 starts at 404 with receiving image data from the localizer 110. The image data includes optical image data and IR image data from the optical camera unit 112 and infrared sensor unit 114, respectively. The image data represents the operating room as captured by the localizer 110. At 408, the image data is normalized. More specifically, the image data may be altered to account for changing environmental conditions in the operating room, such as a change in the brightness of the room. As described in more detail below, normalizing the image may be necessary to ensure that the parameters of the detected objects are not affected by environmental conditions.

At 412, the image data is input to the object detector 312 and the object detector 312 attempts to detect any/all objects present in the image data. Assuming that objects are detected in the image data, the method continues to 416. At 416, the feature extractor 314 and the motion detector 316 are applied to the image data. Based on the outputs from the feature extractor 314 and the motion detector 316, the system 100 determines the parameter(s) associated with the object(s) detected in the image data.

At 418, the system 100 compares the parameters determined at 416 against any existing temporary descriptors. If there are no existing temporary descriptors (e.g., this is the first object introduced to the system 100), the step at 418 may be skipped instead. The comparison at 418 may include a comparison of the parameters of the detected object against the parameters included in the existing temporary descriptors associated with each tracking entity 300 stored in the tracking database 318. At 420, the system 100 determines whether the object(s) detected in the image data match any existing tracking entities 300 using the comparison performed at 418. If the parameters of the detected object match the temporary descriptor of one of the existing tracking entities 300, the method proceeds to 424A

At 424A, the existing tracking entity is revived, for example, by the reviving module 322. That being said, if the parameters of the object do not match the parameters of any existing temporary descriptors, the method proceeds to 424B. At 424B, the system 100 creates a new tracking entity 300, including a new temporary descriptor based on the parameters of the detected object, and associates the new tracking entity 300 with said object. If the tracker 150 is coupled to the object, the new tracking entity 300 also contains the relationship between the object/temporary descriptor and the tracker 150. This step may include detecting the pose of the tracker 150, which allows the system 100 to correlate the pose of the tracker 150 with the pose of the temporary descriptor. And to determine the pose of the tracking entity 300 by determining the pose of the tracker 150. In some implementations, the tracking database is cleared at the end of each use of the system 100. In these implementations, on the first iteration of the method 400, the determination at 420 is skipped and the method 400 instead proceeds from 416 to 424B.

The steps at 424A and 424B may be performed in different ways depending on the configuration of the navigation system 100. In one implementation, the system 100 automatically attempts to create a new tracking entity for a detected object if the parameters of the detected object do not match that of any existing tracking entities. In other implementations, the system 100 waits for a triggering condition before attempting to create a new tracking entity. In one example, the triggering condition is an input from the user. In this example, the system 100 may determine that the parameters of the object do not match any existing tracking entities and thus a new tracking entity should be created. However, instead of automatically creating the new tracking entity, the system 100 waits for the input from the user before creating the new tracking entity for the object. Alternatively, the triggering condition may be the placing of the object at a specific position relative to the localizer 110. In this example, the system 100 would not attempt create a new tracking entity unless the object was at said position. More specifically, the position may be on a table within view of the localizer 110. Here, the system 100 may detect the object and its parameters at 412 and 416, but only create a new tracking entity if the object is perceived by the localizer 110 as being on the table.

After 424A or 424B, the method 400 proceeds to call the tracking manager 320 at 428 followed by the navigation manager 324 at 432. The navigation system 100 utilizes the tracking manager 320 to manage the active and inactive tracking entities 300. The navigation system 100 utilizes the navigation manager 324 to manage how the surgical objects are tracked by the localizer.

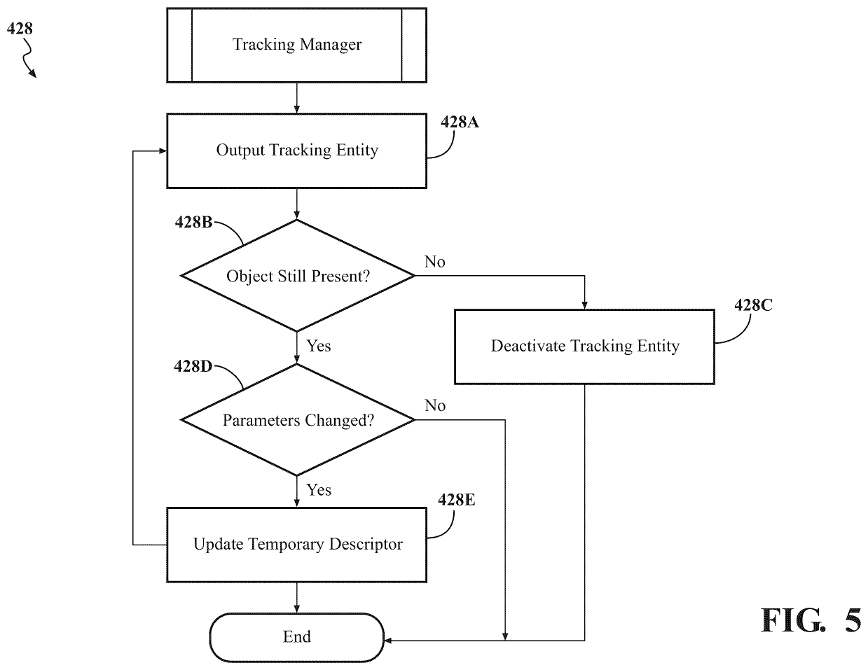

Referring to FIG. 5, an example process performed by the tracking manager 320 during the method 400 is shown. In the example process, the tracking manager 320 is called at 428 during the method 400 and starts by outputting the tracking entity 300 at 428A. The output tracking entity 300 is either the tracking entity revived at 424A or the tracking entity 300 created at 424B. In either case, the tracking entity is output along with a unique identifier such that each tracking entity 300 can be distinguished from other tracking entities 300 by the user (and the system 100) according to the unique identifiers assigned to each entity 300. The process then proceeds to 428B.

At 428B, the tracking manager 320 determines if the object is still present in the image data. If not, the process proceeds to 428C at which point the tracking entity 300 associated with the object is deactivated and the process called at 428 ends. If the object is still present in the image data, the process continues to 428D.

At 428D, the tracking manager 320 determines if any of the parameters of the object have changed over time. For example, the tracking manager 320 may determine that the color of at least part of the object has changed since the tracking entity 300 associated with the object was created. In not, the process ends. If the parameters have changed, the process moves to 428E. At 428E, the temporary descriptor associated with the tracking entity 300 is updated based parameter changes determined at 428D. This allows the navigation system 100 to continue tracking objects in the operating room even after they are altered. For example, if the object is the surgical instrument 250, the instrument 250 may become partially covered in blood during an operation. Updating the color parameter of the tracking entity 300 associated with the instrument 250 may be necessary for the system 100 to be able to recognize the instrument 250 as it becomes discolored by blood or other contaminates. The process then ends after the tracking entity 300 is updated.

The method 400 is depicted in a linear manner, with each step/process occurring one after another, and is generally described as though only one object is detected and tracked at a time. However, the method 400 need not be performed as illustrated. For example, the system 100 may determine that multiple objects are present in the image data at 412. As such, the system 100 may determine parameters for each of the objects and then attempt to match each of the objects to existing tracking entities 300 before continuing to 428. In another example, the tracking manager continuously runs alongside the rest of the method 400. In other words, the method 400 may proceed to 428 as illustrated and then continue to loop through the tracking manager process while also performing a loop of the steps shown at 404 through 424B. This allows the system 100 to control the tracking entities 300 with the tracking manager 320 and remain receptive to characterizing and tracking new objects that become present in the image data at the same time.

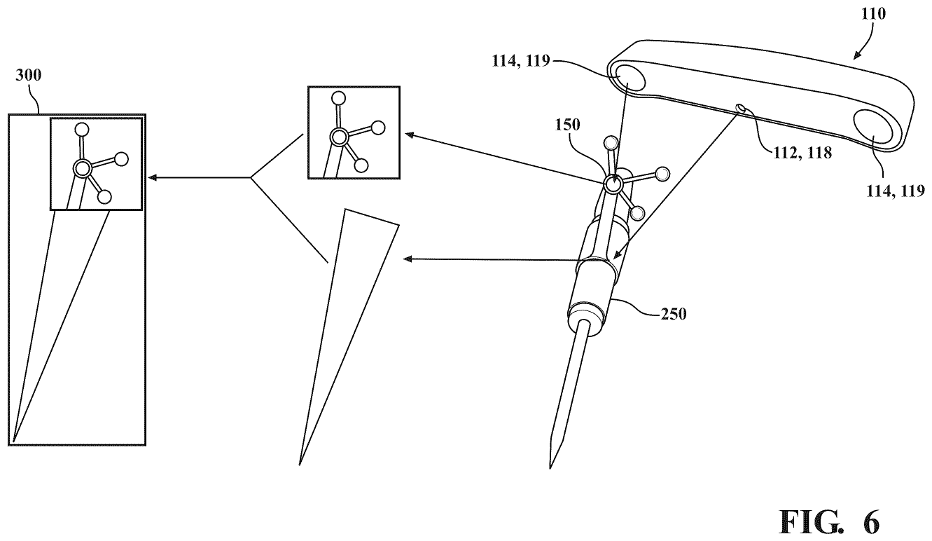

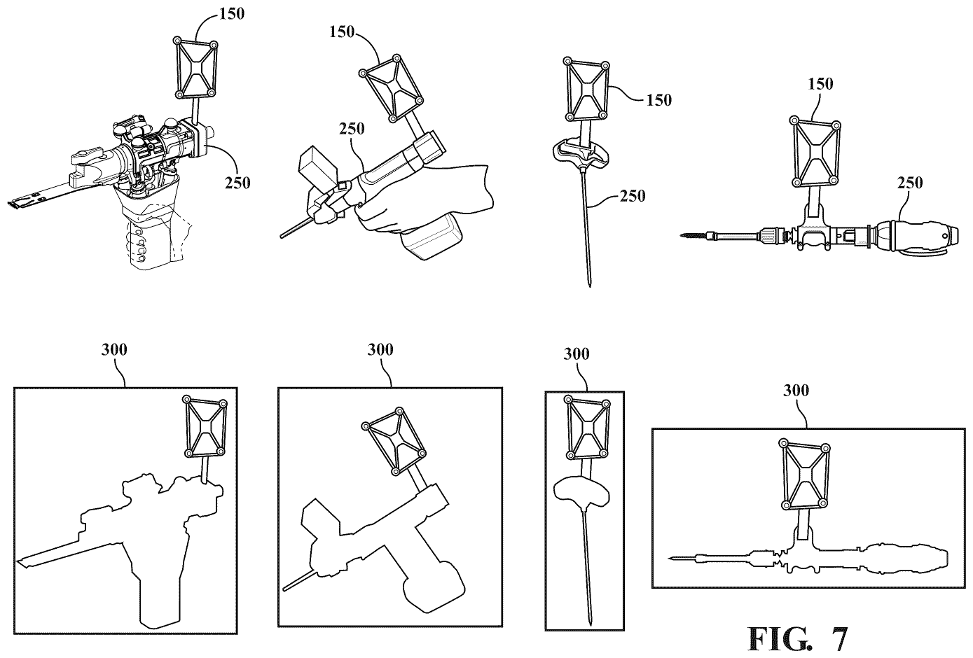

Referring to FIGS. 6 and 7, examples of the tracking entity 300 as created by the navigation system 100 are shown. In the illustrated implementations, the object is the surgical instrument 250 held by the user. The surgical instrument 250 further includes one of the trackers 150 coupled thereto. As described above, the localizer 110 includes an optical camera unit 112 and an IR sensor unit 114 such that the localizer 110 can detect objects in an optical coordinate system and trackers 150 in an IR coordinate system (and/or the optical coordinate system). The navigation controller 104 has access to data pertaining to these coordinate systems such that the controller 104 may register the optical coordinate system to the IR coordinate system. The navigation controller 104 may also register both coordinate systems to a global coordinate system. The localizer 110 detects the pose of the tracker 150 in the IR coordinate system (using any suitable method) and the parameter of the instrument 250 in the optical coordinate system according to the method 400 described above. In the implementation shown, the parameter is simply the shape of the instrument 250 and FIG. 7 includes various tracking entities created based on the respective tracker 150 and shape of the instrument 250.

Once the system 100 has determined the pose of the tracker 150 and the parameter of the instrument 250, the tracker 150 is associated with the instrument 250 and the parameter. This association is stored in the tracking database 318 as the tracking entity 300. After the tracking entity 300 is created for the surgical instrument 250, the pose of the instrument 250 is tracked based on the pose of the tracking entity 300, and the pose of the tracking entity 300 is tracked based on the pose of the tracker 150.

The tracking entities 300 shown in FIGS. 6 and 7 may be used by the user to track the pose of the instrument 250 relative to the patient and/or other objects present in the operating room. For example, an x-ray image of the patient may be shown on one of the displays 106, 107 and the tracking entity 300 may be overlayed as a computer graphic on top of the x-ray image. The computer graphic may be based on the parameters determined by the system 100, and the graphic may include the unique identifier assigned to the tracking entity 300. In such an example, the position of the tracking entity 300 on the display 106, 107 is based on the position of the surgical instrument 250 relative to patient as captured by the localizer 110.

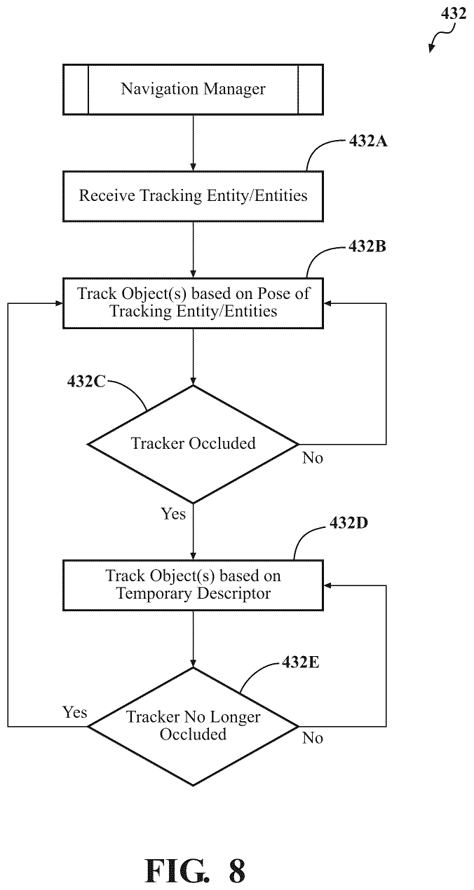

Referring to FIG. 8, an example process performed by the navigation manager 324 during the method 400 is shown. In the example process, the navigation manager 324 is called at 432 during the method 400 and starts by receiving the tracking entity 300 (or entities) at 432A. The navigation manager 324 is generally configured to control how the system 100 tracks the surgical objects using the visible light and IR sensors 118, 119 of the localizer 110. More specifically, since the localizer 110 is capable of tracking the pose of trackers 150 in the IR coordinate system using the IR sensors 119 and the parameters of the surgical object in the optical coordinate system using the visible light sensor 118, the navigation manager 324 controls the localizer 110 and/or navigation controller 104 to rely on a different combination of tracking modalities (e.g. optical and infrared) in order to track the object. The illustrated process shown in FIG. 8 is one process by which the navigation manager 324 may accomplish this multi-modal tracking.

After the tracking entity 300 is received at 432A, the process continues to 432B. At 432B, the navigation manager 324 instructs the navigation controller 104 to track the surgical object based on the pose of the tracking entity 300 output by the tracking manager 320. For example, the navigation controller 104 may track the object based on the pose of the tracker 150. Alternatively, the controller 104 may track the object based on the pose of the temporary descriptor (e.g., the pose of a shape parameter included in the temporary descriptor). Further, the controller 104 may track the object based on a combination of the pose of the tracker 150 and the pose of the temporary descriptor. Even further, the controller 104 may track the object based on a combination of the pose of the tracker 150 and a known relationship between the tracker 150 and the temporary descriptor. The process then proceeds to 432C.

At 432C, the navigation manager 324 determines if the tracker 150 associated with the tracking entity 300 has been occluded from view of the IR sensors 119 of the localizer 110. If not, the process returns to 432B and the system 100 continues tracking the object as normal. However, if the navigation manager 324 determines that the tracker 150 of the tracking entity 300 has been occluded from view, the process moves to 432D. At 432D, the navigation manager 324 begins tracking the object(s) based solely on the temporary descriptor associated with the tracking entity 300 as detected by the visible light sensors 118 of the localizer 110. In other words, the system 100 switches from tracking the object using data from both of the optical and infrared coordinate systems to relying on the data from the optical coordinate system only.

After the system 100 begins tracking the object based on the temporary descriptor (i.e., based on the parameters detected by the visible light sensors 118), the process moves to 432E. At 432E, the navigation manager 324 determines if the tracker 150 is once again detectable by the IR sensors 119. If not, the process moves back to 432D and the system 100 continues tracking the object via the parameters as detected by the visible light sensors 118, but not the pose of the tracker 150 since the tracker 150 is occluded from view of the IR sensors 119. If the tracker 150 is detected at 432E, however, the process returns to 432B and the system 100 switches from tracking the object using data from the optical coordinate system alone, to relying on data from both of the optical and infrared coordinate systems. This may include, for example, reviving the tracking entity using the revival module 322.

The navigation manager 324 thus allows the navigation system 100 to continue tracking the objects in the operating room even if the tracker 150 becomes occluded from view of the localizer 110. In some implementations, after the tracker 150 becomes occluded, the navigation controller 104 may confirm that the surgical object, which is no longer associated with the tracker 150 because it is occluded from view, corresponds to one of the active tracking entities 300. For example, the navigation controller 104 may determine a new parameter(s) of the surgical object between 432C and 432D and compare the new parameter(s) to the temporary descriptor of each active tracking entity 300. If the new parameter(s) matches at least one of the parameters included in the temporary descriptor of one of the active tracking entities 300, the navigation controller 104 may determine that the object corresponds to that tracking entity 300.

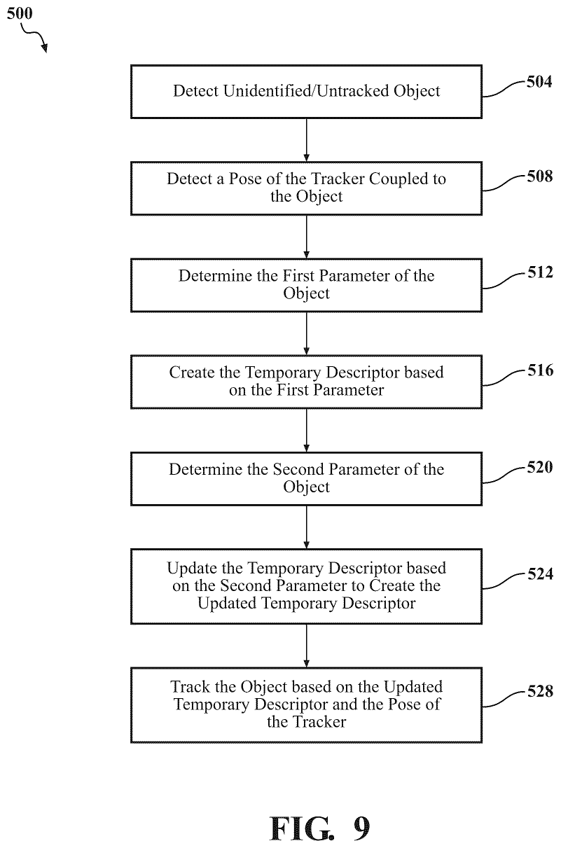

Referring to FIG. 9, an example method 500 of tracking objects in the operating room is depicted. The method 500 is a simplified version of the method 400 shown in FIG. 4, and FIG. 9 is provided to depict a simplified illustration of the method 400 as shown in FIGS. 4, 5, and 8. In the simplified version, the system 100 is presumed to have determined that either the database 318 does not contain any tracking entities 300 (e.g. upon startup of the system 100), or that the parameters of the object do not match any tracking entities 300 stored in the database 318. As such, referring back to the method 400 shown in FIG. 4, the system 100 has already performed at least some of steps 404 through 420 and determined that the object is unidentified and/or untracked. Although the order of certain steps appears different in the method 500 of FIG. 9 form the method 400 of FIGS. 4, 5, and 8, the steps of either method 400, 500 may be performed in any suitable order.

At 504, the navigation controller 104 determines that an unidentified/untracked object has been detected by the localizer 110. This is effectively what occurs at 420 in FIG. 4 when the controller 104 determines that the object does not match any existing tracking entities 300. The method 500 then continues to 508. At 508, the controller detects the pose of the tracker 150 coupled to the object and the method 500 continues to 512.

At 512, the navigation controller 104 determines the first parameter of the object, such as by receiving the first parameter from the localizer 110. After determining the first parameter, the method 500 moves to 516 where the controller 104 creates the temporary descriptor based on the first parameter. At this point, the controller 104 may instruct the system 100 to track the object according to the temporary descriptor and/or the pose of the tracker 150. Otherwise, the method 500 continues to 520 and determines the second parameter of the object. After determining the second parameter, the method 500 moves to 524.

At 524, the controller 104 updates the temporary descriptor 300 based on the second parameter determined at 520 and creates the updated temporary descriptor. Subsequently, at 526, the controller 104 instructs the system to track the object based on the updated temporary descriptor and the pose of the tracker 150. If the object does not have the tracker 150 coupled thereto, any usage of the pose of the tracker 150 in the method 500 may be ignored/skipped. In either case, the step at 528 is substantially similar to the step at 432B of the method 400.

The systems and methods described herein may be partially or fully implemented as instructions stored on a non-transitory computer readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The systems and methods described herein may be partially or fully implemented as computer program products. The computer program products may include processor-executable instructions that are stored on at least one non-transitory computer-readable medium. The computer program products may also include or rely on stored data. The computer program products may encompass a basic input/output system (BIOS) that interacts with hardware of the special purpose computer, device drivers that interact with particular devices of the special purpose computer, one or more operating systems, user applications, background services, background applications, etc. The computer program products may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Several implementations have been discussed in the foregoing description. However, the implementations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A surgical navigation system comprising:
a tracker coupled to a surgical object and comprising tracking elements arranged in a tracker geometry;
a localizer configured to track the tracker and to detect parameters of the surgical object; and
a controller in communication with the localizer and configured to:
detect, with the localizer, a pose of the tracker geometry,
receive a first parameter of the surgical object detected by the localizer,
create, based on the first parameter, a temporary descriptor describing an appearance of the surgical object,
receive a second parameter of the surgical object detected by the localizer,
update, based on the second parameter, the temporary descriptor to create an updated temporary descriptor describing the appearance of the surgical object, and
track the surgical object based on a combination of the updated temporary descriptor and the pose of the tracker geometry.

2. The surgical navigation system of claim 1, wherein:
the first parameter is detected by the localizer at a first time;
the second parameter is detected by the localizer at a second time; and
the first time is prior to the second time.

3. The surgical navigation system of claim 1, wherein:
at least one of the first and second parameters is a physical parameter of the surgical object; and
the physical parameter includes at least one of a geometry or shape of the surgical object, a contour of the surgical object, a color of the surgical object, an envelope of the surgical object, a surface roughness of the surgical object, a surface marking of the surgical object, or a color or shading of the surgical object.

4. The surgical navigation system of claim 1, wherein:
at least one of the first and second parameters is a motion parameter of the surgical object; and
the motion parameter includes at least one of a speed or velocity of the surgical object, an acceleration of the surgical object, a rotation of the surgical object, and a displacement of the surgical object.

5. The surgical navigation system of claim 1, wherein the controller is configured to detect an environmental condition and to normalize the detected parameters according to the environmental condition.

6. The surgical navigation system of claim 1, wherein:
the localizer includes a first sensor configured to detect visible light and second sensor configured to detect infrared or near-infrared light;
the first parameter is detected by the first sensor;
the pose of the tracker geometry is detected by the second sensor.

7. The surgical navigation system of claim of claim 6, wherein:
the controller detects the pose of the tracker geometry in a first coordinate system and the first parameter in a second coordinate system; and
the controller is configured to register one of the first and second coordinate systems to one of the first coordinate system, the second coordinate system, and a third coordinate system.

8. The surgical navigation system of claim 1, wherein the controller is configured to:
detect a third parameter of the surgical object,
update the updated temporary descriptor based on the third parameter, and
track the pose of the surgical object based on a combination of the updated temporary descriptor and the pose of the tracker geometry.

9. The surgical navigation system of claim 1, wherein the controller is configured to:
determine that the pose of the tracker geometry is not detectable by the localizer, and
track the pose of the surgical object based solely on the updated temporary descriptor in response to determining that the pose of the tracker geometry is not detectable by the localizer.

10. The surgical navigation system of claim 9, wherein the controller is configured to:
determine that the pose of the tracker geometry is detectable by the localizer after determining that the pose of the tracker geometry is not detectable by the localizer, and
track the pose of the surgical object based on at least one of the updated temporary descriptor and the pose of the tracker geometry in response to determining that the pose of the tracker geometry is detectable by the localizer.

11. The surgical navigation system of claim 10, wherein the controller is configured to register or re-register the tracker to the surgical object by comparing a new parameter of the surgical object to the updated temporary descriptor.

12. The surgical navigation system of claim 1, wherein the controller provides the first and second parameters to a machine learning module, and the machine learning module outputs a calculated parameter of the surgical object based on the first and second parameters.

13. The surgical navigation system of 12, wherein the controller is configured to:
update the updated temporary descriptor based on the calculated parameter to create a learned temporary descriptor describing the association between the surgical object and the detected tracker geometry, and
track the pose of the surgical object based on a combination of the learned temporary descriptor and the pose of the tracker geometry.

14. The surgical navigation system of claim 13, wherein the controller is configured to:
receive a third parameter of the surgical object,
compare the calculated parameter to the third parameter,
update the learned temporary descriptor based on the comparison to create an updated learned temporary descriptor describing the association between the surgical object and the detected tracker geometry, and
track the pose of the surgical object based on a combination of the updated learned temporary descriptor and the pose of the tracker geometry.

15. The surgical navigation system of claim 1, wherein the second parameter is realized as a motion parameter, and the controller is configured to:
detect a pose change of the tracker geometry,
create the updated temporary descriptor based on a determination that the motion parameter is sufficiently aligned with the pose change of the tracker geometry.

16. The surgical navigation system of claim 1, wherein the controller is configured to replace the first parameter with the second parameter in response to a determination that the second parameter is inconsistent with the first parameter.

17. The surgical navigation system of claim 1, wherein the tracker is realized as a first tracker and the surgical object is realized as a first surgical object, and further comprising:
a second tracker coupled to a second surgical object and comprising tracking elements arranged in a tracker geometry that is identical to the tracker geometry of the first tracker; and
wherein the localizer is configured to track the first and second trackers and to detect parameters associated with the first surgical object and second surgical object; and
wherein the controller is configured to:
detect, with the localizer, a pose of the first tracker and a first parameter associated with the first surgical object,
create a first tracking entity based on the pose of the first tracker and the first parameter,
detect, with the localizer, a pose of the second tracker and a second parameter associated with the second surgical object,
create a second tracking entity based on the pose of the second tracker and the second parameter, and
track poses of the first and second surgical objects based on movement of the first and second tracking entities, respectively.

18. The surgical navigation system of claim 1, further comprising:
a database containing descriptors of surgical objects; and
wherein the controller is configured to:
compare the first parameter of the surgical object to the descriptors stored in the database,
based on the comparison, determine that there are no descriptors in the database describing the appearance of the surgical object, and, in response, create the temporary descriptor describing the surgical object based on the detected parameter of the surgical object.

19. A method of tracking a surgical object using a localizer, the surgical object being coupled to a tracker comprising tracking elements arranged in a tracker geometry, the method comprising:
detecting, with the localizer, a pose of the tracker geometry,
receiving a first parameter of the surgical object detected by the localizer,
creating, based on the first parameter, a temporary descriptor describing an appearance of the surgical object,
receiving a second parameter of the surgical object detected by the localizer,
updating, based on the second parameter, the temporary descriptor to create an updated temporary descriptor describing the appearance of the surgical object, and
tracking the surgical object based on a combination of the updated temporary descriptor and the pose of the tracker geometry.

20. A non-transitory computer readable medium having instructions stored thereon, the instructions configured to be executed by a controller connected to a localizer to cause the controller to perform operations comprising:
detecting, with the localizer, a pose of the tracker geometry,
receiving a first parameter of the surgical object detected by the localizer,
creating, based on the first parameter, a temporary descriptor describing an appearance of the surgical object,
receiving a second parameter of the surgical object detected by the localizer,
updating, based on the second parameter, the temporary descriptor to create an updated temporary descriptor describing the appearance of the surgical object, and
tracking the surgical object based on a combination of the updated temporary descriptor and the pose of the tracker geometry.
